(19) United States
(12) Patent Application Publication
(10) Pub. No.: US 2024/0201300 A1
(43) Pub. Date: Jun. 20, 2024

(54) METHOD FOR DETERMINING A PARAMETER SETTING FOR A GRADIENT POWER OF A MAGNETIC RESONANCE SYSTEM, COMPUTER PROGRAM PRODUCT, COMPUTER-READABLE STORAGE MEDIUM AND ELECTRONIC COMPUTING DEVICE

(71) Applicant: Siemens Healthcare GmbH, Erlangen (DE)

(72) Inventors: Max Müller, Erlangen (DE); Mario Zeller, Erlangen (DE); Adam Kettinger, Erlangen (DE); Dominik Paul, Bubenreuth (DE); Thorsten Feiweier, Poxdorf (DE); Gudrun Ruyters, Erlangen (DE); Michael Köhler, Nürnberg (DE)

(21) Appl. No.: 18/522,591

(22) Filed: Nov. 29, 2023

(30) Foreign Application Priority Data
Dec. 19, 2022 (DE) 10 2022 213 903.8

(51) Int. Cl. G01R 33/54; G01R 33/28

(52) U.S. Cl. CPC G01R 33/543; G01R 33/288; A61B 5/055

(57) ABSTRACT

A method for determining a parameter setting for a gradient power of a magnetic resonance system by an electronic computing device. The method includes specifying a limit value for a nerve stimulation in the case of a person positioned in the magnetic resonance system, entering at least one gradient parameter for a pulse of the gradient power as the parameter setting by an input device of the electronic computing device, approximating a potential nerve stimulation as a function of the at least one gradient parameter by a predefined mathematical model of the electronic computing device, comparing the approximated potential nerve stimulation with the predefined limit value by the electronic computing device, and determining the parameter setting as a function of the comparison.

### CROSS REFERENCE TO RELATED APPLICATIONS

This application claims the benefit of DE 10 2022 213 903.8, filed on Dec. 19, 2022, which is hereby incorporated by reference in its entirety.

### FIELD

Embodiments relate to a method for determining a parameter setting for a gradient power of a magnetic resonance system by an electronic computing device.

### BACKGROUND

A constraining factor in the application of magnetic resonance tomography (MRT) to human subjects in systems providing strong gradient performance are the limit values for potential peripheral nerve stimulation (PNS) or cardio nerve stimulation (CNS). Selected sequence protocols may not be performed due to overly rapid magnetic field variations. Whether a sequence may be executed is checked before the start of the measurement in a sequence check method by way of a corresponding framework of the control software, after the user or operator has actuated the sequence start. If it is established in this check that the resulting gradient sequence would reach stimulation values that would exceed the corresponding limit values, a search is conducted for a new parameter configuration (solver) and this is proposed to the user, thereby keeping the examination method below the limit values for the stimulation. A key parameter for this is a reduction in what is termed the gradient rise time (RT) or ramp time, the multiplicative inverse of what is referred to as the slew rate (SR) or rate of rise.

A problem is that the framework solver does not return which gradient triggered the stimulation and may only use a single reduced rise time for the solution search. Consequently, with more complex sequences (for example EPI diffusion), it is not possible to consider different parts of the sequence in a differentiated manner or to resolve simulation conflicts. If one part stimulates, for example the diffusion part, the reduction in the global rise time will also unnecessarily limit the EPI readout, and vice versa. This has led in the past to fixed “worst case” rise times being used for the diffusion part, by which a stimulation is highly improbable, and the framework solver was then applied only to the EPI readout parts. However, this caused much of the potential gradient power to be wasted in the diffusion part. This problem intensifies with the increasing gradient performance offered by the new magnetic resonance systems.

A further problem with the framework check is that the latter is not initiated until after the start of the measurement has been confirmed, so that initially the user possibly selects inconsistent protocols which additionally wastes time in order to resolve these again accordingly. Also, the framework check conducts a test numerically in small time increments, that may lead to long calculations if the time interval to be checked is long.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art.

Embodiments provide a method, a computer program product, a computer-readable storage medium, and an electronic computing device by which a parameter setting for a gradient power of a magnetic resonance system may be determined.

Embodiments provide a method for determining a parameter setting for a gradient power of a magnetic resonance system by an electronic computing device. A limit value is specified for a nerve stimulation in the case of a person disposed in the magnetic resonance system. At least one gradient parameter for a pulse of the gradient power is entered as the parameter setting by an input device of the electronic computing device. A potential nerve stimulation is approximated as a function of the at least one gradient parameter by a predefined mathematical model of the electronic computing device. The approximated potential nerve stimulation is compared with the predefined limit value by the electronic computing device and the parameter setting is determined once again as a function of the comparison.

A simplified approximation of the mathematical model is applied already by way of a help function in the sequence-prepare phase, in other words already at the time of processing the measurement protocol, for individual ramps, gradients or gradient sequences identified as critical at the preliminary stage, in order to examine these for stimulation. In the sequence-prepare phase, checks are carried out continuously during the user input to determine whether a valid parameter combination is present, and the user is allowed to input valid combinations only. A sequence check is initiated after the user has pressed “Start”. If an invalid sequence configuration is found here, a solution assistant (solver) attempts to suggest a valid alternative to the user. Alternatively, the user may go back to the protocol processing step in order to input a different configuration in person. In the case of a diffusion sequence, for example, this may be a sequence of diffusion encoding gradients having the highest weighting factor or b-value or a high gradient moment on the readout axis. In addition or alternatively, attention may be focused for example on gradients on that axis that exhibit the greatest stimulation potential.

The method permits a dynamic, for example iterative, adjustment of individual critical gradient sections. This provides individual stimulating diffusion gradients to be adjusted for example by alternating the check with the presented mathematical model and for example by reducing the gradient amplitude and/or rise time until no stimulation occurs any longer. Previously greater time intervals were examined based on numeric calculations, that is time-consuming and therefore makes iterative adjustment impractical.

This method may be used productively on systems delivering very high gradient performance. It has been shown in this context that the readout train is usually limited due to peripheral nerve stimulation (PNS), high diffusion gradients in contrast due to cardio nerve stimulation (CNS). By checking the diffusion gradients for CNS stimulation, for example using the corresponding constants relevant to CNS, and corresponding reduction of the gradient parameters, a subsequent application of the SAFE model to the readout train may only lead to a resolution of potential PNS conflicts. An unnecessary power reduction due to the inability to distinguish between time intervals susceptible to CNS or to PNS is avoided, in contrast to the conventional approach in which the slew rate might only be adjusted globally.

For example in the case of diffusion measurements, data including different weightings (b-values) and directions is generally acquired sequentially. To speed up the presented method further, the stimulation check for example, for example, may also be carried out directly in the sequence-prepare phase and is therefore fast, utilizes the gradients for each protocol to the maximum within the scope of the limits, and permits the user, for example the user of the magnetic resonance system, to set valid values only. The gradient power is used in an improved manner as a result. Furthermore, the time for protocol settings or corrections, and consequently the patient table time, is reduced.

For example a peripheral nerve stimulation and a cardio nerve stimulation may therefore be considered as nerve stimulation. For example, a measurement protocol may be specified by a user, for example resolution, position, and dimensions of the slices (diffusion encoding parameters) and the electronic computing device or the input device “translates” these requirements into a sequence of gradient and RF pulses. The gradient pulses are characterized by gradient parameters (for example the slew rate).

The approximation is performed for example on time increments, defined by start and end points of piecewise linear gradient amplitude curves. Only by the presented analytical calculation of longer time intervals in a computational step is it possible to “calculate” fast enough to enable these calculations to be performed continuously during the user input already in the sequence-prepare phase.

A numeric calculation in small time increments (prior art) would be too computationally intensive during the user input and may therefore only be performed subsequently in the sequence-check phase.

According to an embodiment, a gradient amplitude of the pulse and/or a slew rate of the pulse are generated as gradient parameters as a function of an input. For example, a user of the magnetic resonance system may set the gradient parameter or the gradient amplitude or the slew rate accordingly, thereby effecting a corresponding adjustment of the imaging method, that is for example a modality referred to as diffusion-weighted imaging. On this basis, it may now be provided that a corresponding evaluation is performed, and it may thus be determined whether the limit value for the nerve stimulation is reached in the case of the gradient amplitude or the slew rate.

A nerve stimulation formed in all three spatial directions as the potential nerve stimulation may be approximated as the total nerve stimulation. For example, it may be provided for example that the total nerve stimulation Nₜₒₜₐₗ is determined from the formula:

$${N}_{Total} = \sqrt{{N}_{x}^{2} + {N}_{y}^{2} + {N}_{z}^{2}} .$$

Nx, Ny and Nz are the individual nerve stimulations in the three spatial directions x, y and z. Shown by way of example for x, the simulation of an axis is thereby calculated from the sum of three filter functions F for example:

$${N}_{x} = \frac{( {F}_{1 , x} + {F}_{2 , x} + {F}_{3 , x} )}{{L}_{x}} \times {gs}_{x}$$

where Lₓ is the stimulation limit of the corresponding physical gradient axis and gsₓ is an axis-specific gradient scaling factor, that is obtained for example on a system-specific basis from a database. The three filter functions F are calculated as follows, where τ is the respective attenuation constant:

$${F}_{1 , x} = {a}_{1 , x} \vert f ( {S}_{x} , T , {τ}_{1 , x} ) \vert$$

$${F}_{2 , x} = {a}_{2 , x} f ( \vert {S}_{x} \vert , T , {τ}_{2 , x} )$$

$${F}_{3 , x} = {a}_{3 , x} \vert f ( {S}_{x} , T , {τ}_{3 , x} ) \vert$$

For simple linear ramp/plateau sections, as are given for example in established diffusion encoding sequences, the filter kernel f may be approximated to:

$${f}_{n} = {f}_{n - 1} {e}^{- \frac{{T}_{n}}{{τ}_{c}}} + {S}_{n} ( 1 - {e}^{- \frac{{T}_{n}}{{τ}_{c}}} )$$

where f₀=0 or is an assumed “initial stimulation” from preceding events. A step n in this case describes a time duration T with constant gradient slew rate Sₙ (RampUp (ramp-up), FlatTop (plateau) or RampDown (ramp-down)). τ_{c} describes the respective corrected attenuation constant. It is possible for the potential nerve stimulation to be determined in a simple manner.

A respective nerve stimulation may be determined in all three spatial directions and the potential nerve stimulation may be approximated as that which has the highest value of the three nerve stimulations in one spatial direction. As a result, for example in order to accelerate the presented method further, the calculation may be restricted to a subset of the direction-weighting combination occurring for the maximum stimulation level. For example, the amplitude of the diffusion gradients scales with the root of the b-value. The maximum stimulation levels consequently occur at the highest b-value. It may therefore suffice to consider only the acquisition having the maximum b-value. For example, the different gradient axes have different degrees of stimulation potential, for example relative to the orientation of the patient. For example in the case of acquisitions having a number of isotropic diffusion directions, it may therefore be sufficient to consider that direction having the maximum component along the axis with the highest simulation potential.

The mathematical model may be specified as an analytical mathematical model. For example, therefore, the mathematical model is not a numeric model. A ramp-up of the pulse and/or a plateau of the pulse and/or a ramp-down of the pulse are/is evaluated analytically. For example for simple linear ramps or plateau sections within the pulse, as are given for example in the case of the established diffusion encoding sequences, a corresponding filter kernel f may be approximated to:

$${f}_{n} = {f}_{n - 1} {e}^{- \frac{{T}_{n}}{{τ}_{c}}} + {S}_{n} ( 1 - {e}^{- \frac{{T}_{n}}{{τ}_{c}}} )$$

where f₀=0 or is an assumed “initial stimulation” from preceding events. A step n in this case describes a time duration T with constant gradient slew rate Sₙ (RampUp (ramp-up), FlatTop (plateau) or RampDown (ramp-down)). τ_{c} describes the respective corrected attenuation constant.

The predefined limit value may be specified with a safety factor. For example, this provides a simple check to be conducted by the mathematical model in the sequence-prepare phase to determine whether the total stimulation Nₜₒₜₐₗ for individual gradient objects exceeds the limit value of 100 percent and a search to be conducted for a direct solution. A safety factor may be introduced on account of approximation assumptions or preconditions. For example, only 95 percent of the maximum total stimulation may be specified as the limit value in this case.

The parameter setting may be determined in real time. For example, the parameter setting is determined substantially in real time. In this way it is made possible for the user of the magnetic resonance system or of the electronic computing device to obtain the corresponding limits on measurement parameter values in real time and, for example, to see whether limit values are being exceeded.

A potential peripheral nerve stimulation and/or a potential cardio nerve stimulation may be taken into account in the determining of the parameter setting. For example it is provided that no exceeding of the predefined limit values for cardiac stimulation may occur. For example, this provides the limit values to be adjusted accordingly already before the actual determination method so that, for example, parameter settings that would cause a cardio nerve stimulation are already suppressed and are not available for selection.

Limit values for the peripheral nerve stimulation and/or the cardio nerve stimulation may be specified for determining the parameter setting. This provides the sequence to be subdivided into sections, of which it is known that either only CNS or only PNS are relevant and therefore only a test for one or the other must be carried out. The sequence may also be subdivided into sections in which the slew rate or amplitude is adjusted on a “partially global” basis in order to limit PNS and/or CNS.

Only one gradient parameter within a predefined range for the pulse may be allowed as input by the electronic computing device. For example, this provides gradient parameters that, for example, would also apply to a cardio nerve stimulation to be suppressed and not be provided to the user for selection. For example, parameters are allowed only when a corresponding solution is possible within the limit values. This speeds up the method for the user and at the same time input errors may be prevented.

The presented method may be a computer-implemented method. Embodiments further provide a computer program product including program code that cause an electronic computing device to perform a method according to the preceding aspect when the program code are processed by the electronic computing device.

Embodiments further provide a computer-readable storage medium containing a computer program product according to the preceding aspect.

Embodiments further provide an electronic computing device for a magnetic resonance system for determining a parameter setting for a gradient power of the magnetic resonance system, the device including at least one input device. The electronic computing device is configured for performing a method according to the preceding aspect. For example, the method is performed by the electronic computing device.

The electronic computing device has for example processors, circuits, for example integrated circuits, as well as further electronic components in order to be able to perform corresponding method steps.

Further, embodiments also relates to a magnetic resonance system including an electronic computing device according to the preceding aspect.

Advantageous embodiments of the method are to be regarded as advantageous embodiments of the electronic computing device as well as of the magnetic resonance system. To that end, the electronic computing device and the magnetic resonance system possess object-related features in order to be able to perform corresponding method steps.

For application cases or application situations that may arise with the method and that are not explicitly described here, it may be provided according to the method that an error message and/or a request to input a user feedback response are/is output and/or a default setting and/or a predetermined initial state are/is set.

Independent of the grammatical term usage, individuals with male, female or other gender identities are included within the term.

### BRIEF DESCRIPTION OF THE FIGURES

FIG. 1 depicts a schematic block diagram according to an embodiment of a magnetic resonance system including an embodiment of an electronic computing device.

### DETAILED DESCRIPTION

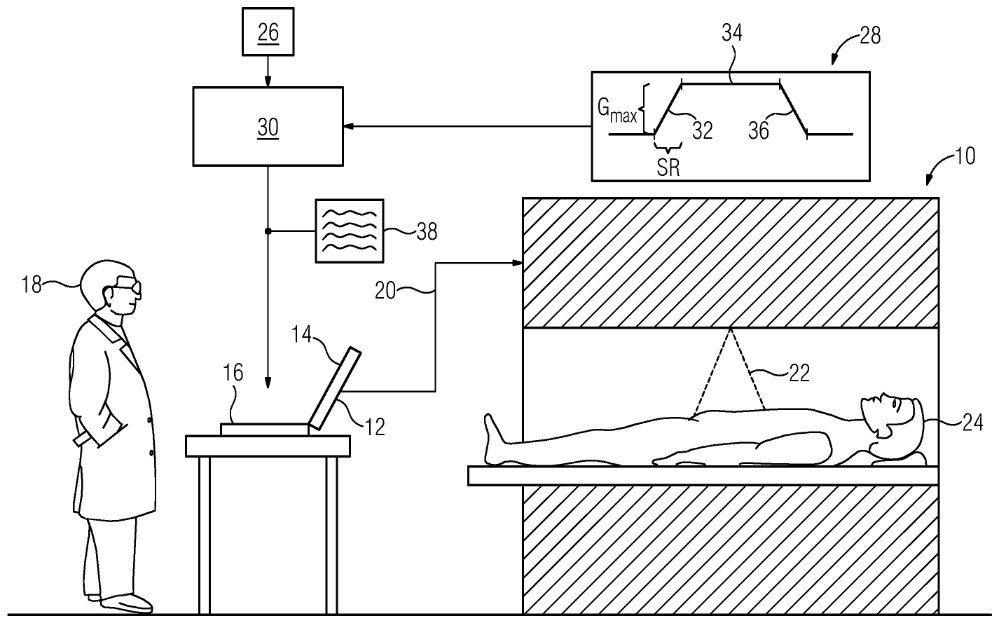

FIG. 1 depicts a schematic block diagram of an embodiment of a magnetic resonance system 10. The magnetic resonance system 10 has at least one electronic computing device 12 that, in the present exemplary embodiment, is shown for example as a computer. The computer 12 has for example a display device 14, as well as an input device 16, for example in the form of a keyboard. An operator 18 may in turn select a corresponding parameter setting 20, the parameter setting 20 being dependent for example on an input gradient parameter SR, G.

The magnetic resonance system 10 is configured for example for emitting gradient power 22 in order for example to enable an imaging method to be performed for an examination subject, for example a person 24.

In the presented method it is provided for example that the parameter setting 20 is determined as appropriate. To that end, a limit value is specified for a peripheral and/or cardio nerve stimulation at a check point 24 in the magnetic resonance system 10. The at least one gradient parameter SR, G is entered by the input device 16 for a pulse 28, for example a gradient pulse, of the gradient power 22 as the parameter setting 20. A potential nerve stimulation is approximated as a function of the at least one gradient parameter G, SR by a predefined mathematical model 30 and the approximated potential nerve stimulation is compared with the predefined limit value 26 by the electronic computing device 12 and the parameter setting is also determined as a function of the comparison.

FIG. 1 depicts for example that an input of a gradient amplitude G of the pulse 28 and/or an input of a slew rate SR (rate of rise) of the pulse 28 is acquired as the gradient parameter SR, G. It may further be provided that, for example, a ramp-up 32 of the pulse 28 and/or a plateau 34 of the pulse 28 and/or a ramp-down 36 of the pulse 28 are/is evaluated analytically.

Embodiments provide for example that a simplified approximation of a mathematical model is applied by way of a help function already in the sequence-prepare phase for individual ramps, gradients or gradient sequences identified as critical at the preliminary stage, in order to examine these for stimulation. In the case of the diffusion sequence, this may be for example a sequence of diffusion encoding gradients with the highest b-value or with a high gradient moment on the readout axis. In addition or alternatively, consideration may be given for example to gradients on that axis that shows the greatest potential for stimulations.

The total nerve stimulation N_{Total} relevant to the check is calculated from the contributions of the individual gradient axes:

$${N}_{Total} = \sqrt{{N}_{x}^{2} + {N}_{y}^{2} + {N}_{z}^{2}}$$

Shown by way of example for x, the stimulation of an axis is calculated from the sum of three filter functions F:

$${N}_{x} = \frac{( {F}_{1 , x} + {F}_{2 , x} + {F}_{3 , x} )}{{L}_{x}} \times {gs}_{x}$$

where Lₓ is the stimulation limit of the corresponding physical gradient axis and gsₓ is an axis-specific gradient scaling factor.

The three filter functions F are calculated as follows, where τ is the respective attenuation constant:

$${F}_{1 , x} = {a}_{1 , x} \vert f ( {S}_{x} , T , {τ}_{1 , x} ) \vert$$

$${F}_{2 , x} = {a}_{2 , x} f ( \vert {S}_{x} \vert , T , {τ}_{2 , x} )$$

$${F}_{3 , x} = {a}_{3 , x} \vert f ( {S}_{x} , T , {τ}_{3 , x} ) \vert$$

For simple linear ramps/plateau sections, as are given for example in established diffusion encoding sequences, the filter kernel f may be approximated to:

$${f}_{n} = {f}_{n - 1} {e}^{- {T}_{n} / {τ}_{c}} + {S}_{n} ( 1 - {e}^{- {T}_{n} / {τ}_{c}} )$$

where f₀=0 or is an assumed “initial stimulation” from preceding events. A step n in this case describes a time period T having a constant gradient slew rate Sₙ (RampUp, FlatTop or RampDown). τ_{c} describes the respective corrected attenuation constant, where τ_{c}=τ+0.5*GRT is assumed and GRT corresponds to a gradient raster time, i.e. the temporal spacing of the gradient interpolation points.

By this model it may be easily checked in the sequence-prepare phase whether the total stimulation N_{Total} for individual gradient objects exceeds the limit value of 100% (or with safety buffer on account of the approximation assumptions/preconditions for example 95%) and a solution may be searched for directly.

For example, the presented method permits a dynamic (iterative) adjustment of individual critical gradient sections. Thus, individual stimulating diffusion gradients may be adjusted for example by alternating checking with the presented model and with a reduction in the gradient amplitude G and/or slew rate SR until such time as no further stimulation occurs. In the prior art, greater time intervals were previously examined by numeric calculation, that is time-consuming and therefore makes an iterative adjustment impractical.

The method may be used productively on systems delivering very high gradient performance. It has been shown in this context that the readout train is usually limited due to PNS (peripheral nerve stimulation), high diffusion gradients in contrast due to CNS (cardio nerve stimulation). By checking the diffusion gradients for CNS stimulation, using the corresponding constants relevant to CNS, and corresponding reduction of the gradient parameters G, SR, a subsequent application of the SAFE model to the readout train would only lead now to a resolution of potential PNS conflicts. An unnecessary power reduction due to the inability to distinguish between time intervals susceptible to CNS or to PNS is avoided thanks to the SAFE model.

In diffusion measurements, data is usually acquired sequentially using different weightings (b-values) and directions. In order to speed up the presented method further, the calculation may be restricted to a subset of the direction-weighting combinations occurring for the maximum stimulation level.

For example, the amplitude of the diffusion gradients scales with the root of the b-value: maximum stimulation levels consequently occur at the highest b-value. It may therefore suffice simply to consider the acquisitions having the maximum b-value.

Furthermore, the different gradient axes (relative to the orientation of the patient) have for example different degrees of stimulation potential. For example in the case of acquisitions having a number of isotropic diffusion directions, it may therefore be sufficient to consider that direction having the maximum component along the axis with the highest stimulation potential.

As an alternative to the iterative determination of optimized rise times, it is possible by the presented method to determine once only—for example at the time of loading the sequence code—a table containing rise times optimized in each case for different combinations of diffusion directions and gradient amplitudes G, and if applicable for different diffusion encoding schemes, for example “monopolar” or “bipolar” gradient sequences. When the actual rise times for the preparation of a particular measurement protocol are specified, the precalculated values may then be quickly looked up.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining a parameter setting for a gradient power of a magnetic resonance system by an electronic computing device, the method comprising:
specifying a limit value for a nerve stimulation of a person positioned in the magnetic resonance system;
entering at least one gradient parameter for a pulse of the gradient power as the parameter setting by an input device of the electronic computing device;
approximating a potential nerve stimulation as a function of the at least one gradient parameter by a predefined mathematical model of the electronic computing device;
comparing the approximated potential nerve stimulation with the limit value by the electronic computing device; and
determining the parameter setting as a function of the comparison.

2. The method of claim 1, wherein a gradient amplitude of the pulse, a slew rate of the pulse, or the gradient amplitude of the pulse and the slew rate of the pulse are generated as the gradient parameter as a function of an input.

3. The method as of claim 1, wherein, as the potential nerve stimulation, a nerve stimulation formed in all three spatial directions is approximated as a total nerve stimulation.

4. The method of claim 3, wherein a potential nerve stimulation is approximated for each spatial direction and the total nerve stimulation is determined by:
$${N}_{Total} = \sqrt{{N}_{x}^{2} + {N}_{y}^{2} + {N}_{z}^{2}} ,$$
where N_{Total} corresponds to the total nerve stimulation and Nₓ, N_{y}, N_{z} correspond to the respective nerve stimulation in one spatial direction.

5. The method of claim 1, wherein a respective nerve stimulation is determined in all three spatial directions and the potential nerve stimulation is approximated as that which has the highest value of the determined three nerve stimulations in one spatial direction.

6. The method of claim 1, wherein the predefined mathematical model is specified as an analytical mathematical model.

7. The method of claim 6, wherein at least one of a ramp-up of the pulse, a plateau of the pulse, or a ramp-down of the pulse is evaluated analytically.

8. The method of claim 1, wherein the limit value is specified with a safety factor.

9. The method of claim 1, wherein the parameter setting is determined in real time.

10. The method of claim 1, wherein a peripheral nerve stimulation, a potential cardio nerve stimulation, or the peripheral nerve stimulation and the potential cardio nerve stimulation are taken into account in the determination of the parameter setting.

11. The method of claim 10, wherein limit values for the peripheral nerve stimulation, the cardio nerve stimulation, or the peripheral nerve stimulation and the cardio nerve stimulation are specified for determining the parameter setting.

12. The method of claim 1, wherein only one gradient parameter within a predefined range for the pulse is allowed as input by the electronic computing device.

13. A non-transitory computer implemented storage medium that stores machine-readable instructions executable by at least one processor for determining a parameter setting for a gradient power of a magnetic resonance system by an electronic computing device, the machine-readable instructions comprising:
specifying a limit value for a nerve stimulation of a person positioned in the magnetic resonance system;
entering at least one gradient parameter for a pulse of the gradient power as the parameter setting by an input device of the electronic computing device;
approximating a potential nerve stimulation as a function of the at least one gradient parameter by a predefined mathematical model of the electronic computing device;
comparing the approximated potential nerve stimulation with the limit value by the electronic computing device; and
determining the parameter setting as a function of the comparison.

14. The non-transitory computer implemented storage medium of claim 13, wherein a gradient amplitude of the pulse, a slew rate of the pulse, or the gradient amplitude of the pulse and the slew rate of the pulse are generated as the gradient parameter as a function of an input.

15. The non-transitory computer implemented storage medium of claim 13, wherein, as the potential nerve stimulation, a nerve stimulation formed in all three spatial directions is approximated as a total nerve stimulation.

16. The non-transitory computer implemented storage medium of claim 15, wherein a potential nerve stimulation is approximated for each spatial direction and the total nerve stimulation is determined by:
$${N}_{Total} = \sqrt{{N}_{x}^{2} + {N}_{y}^{2} + {N}_{z}^{2}} ,$$
where N_{Total} corresponds to the total nerve stimulation and Nₓ, N_{y}, N_{z} correspond to the respective nerve stimulation in one spatial direction.

17. The non-transitory computer implemented storage medium of claim 13, wherein a respective nerve stimulation is determined in all three spatial directions and the potential nerve stimulation is approximated as that which has the highest value of the determined three nerve stimulations in one spatial direction.

18. The non-transitory computer implemented storage medium of claim 13, wherein the predefined mathematical model is specified as an analytical mathematical model.

19. The non-transitory computer implemented storage medium of claim 18, wherein at least one of a ramp-up of the pulse, a plateau of the pulse, or a ramp-down of the pulse is evaluated analytically.

20. An electronic computing device for a magnetic resonance system for determining a parameter setting for a gradient power of the magnetic resonance system, the device comprising at least one input device, wherein the electronic computing device is configured to:
specify a limit value for a nerve stimulation of a person positioned in the magnetic resonance system;
enter at least one gradient parameter for a pulse of the gradient power as the parameter setting by an input device of the electronic computing device;
approximate a potential nerve stimulation as a function of the at least one gradient parameter by a predefined mathematical model of the electronic computing device;
compare the approximated potential nerve stimulation with the limit value by the electronic computing device; and
determine the parameter setting as a function of the comparison.
